(19) United States
(12) Patent Application Publication
(10) Pub. No.: US 2025/0098941 A1
(43) Pub. Date: Mar. 27, 2025

(54) HAND SURGICAL INSTRUMENT AND BRIDGE FOR A HANDHELD SURGICAL INSTRUMENT

(71) Applicant: OLYMPUS WINTER & IBE GMBH, Hamburg (DE)

(72) Inventors: Irina SCHMUCK, Heide (DE); Kevin Alexander SCHULZ, Wilstedt (DE); Bastian SCHRÖDER, Hamburg (DE); Andreas RÜHS, Ahrensburg (DE)

(73) Assignee: OLYMPUS WINTER & IBE GMBH, Hamburg (DE)

(21) Appl. No.: 18/738,973

(22) Filed: Jun. 10, 2024

Related U.S. Application Data
(60) Provisional application No. 63/540,247, filed on Sep. 25, 2023.

(51) Int. Cl. A61B 1/00; A61B 1/002; A61B 1/018

(52) U.S. Cl. CPC A61B 1/00154; A61B 1/002; A61B 1/018

(57) ABSTRACT

A handheld surgical instrument and a bridge for a handheld surgical instrument, through which bridge a plurality of working instruments can be inserted in an ideal manner into a shaft of the handheld surgical instrument. This is achieved by the fact that a bridge for a handheld surgical instrument has a channel-like guide bore for an optical unit. This bore extends through the entire bridge, specifically from a proximal end to a distal end. The guide bore is segmented from the proximal end to the distal end of the bridge and that the segments have longitudinal axes offset with respect to one another.

The invention relates to a bridge for a handheld surgical instrument according to the preamble of claim 1. The invention further relates to a handheld surgical instrument according to claim 12.

As is known, handheld surgical instruments such as endoscopes, cystoscopes, resectoscopes or the like are used for urological applications. The instruments are guided with a tubular shaft through an opening in the body of the person to be treated and are brought to the position intended for use. Depending on the application or the instrument, various working instruments are guided through the shaft, for example forceps, scissors, clamps or, for example in high-frequency surgery, electric electrodes. In addition, together with the aforementioned working instruments, an optical unit is guided through the shaft into the body in order to achieve visual monitoring of the treatment.

Outside the body, namely at the proximal end of the shaft, there is usually a main body or a bridge of the instrument. Such a bridge usually serves to secure the various working instruments and the optical unit that are to be guided into the shaft. For this purpose, it is provided that valves or other closing or connecting means are arranged on the bridge. It is also conceivable that the bridge has connections for the removal or supply of liquid. Embodiments are also known in which additional electrical or optical instruments can be attached at this bridge. To be able to better operate the instrument along with the bridge and the shaft, it can also have handles.

In these minimally invasive applications, it is of paramount importance that the cross section of the shaft to be introduced into the body is just large enough that all the necessary working instruments can be introduced into the body. Accordingly, the available volume within the shaft must be optimally utilized. It is not only the type and the shape of the instruments to be guided through the shaft that play a role, but also their relative position to one another, specifically over the entire length of the shaft. In particular, very long and very thin instruments, for example a rod lens secured in the bridge and guided through the entire shaft, tilt when the bridge is being coupled to the shaft, so that the space available for the other working instruments is disrupted. Especially when wires are used for the manipulation of levers or forceps, an optical unit that has tilted can be very much a hindrance. In the worst case, the functionality of the working instruments is disturbed by the optical unit tilted in particular in the distal region of the shaft, which can have a negative impact on the result of the application.

The problem addressed by the present invention is therefore to make available a bridge for a handheld surgical instrument, the use of which bridge allows all the working instruments and the optical unit to be guided in an ideal manner into the shaft of the handheld surgical instrument.

A bridge for solving this problem has the features of claim 1. Accordingly, it is provided that a bridge for a handheld surgical instrument, which may be an endoscope, a cystoscope or a resectoscope for example, has a channel-like guide bore for an optical unit. This bore extends through the entire bridge, specifically from a proximal end to a distal end. This bore can be used to guide and, if necessary, also to secure an optical unit, for example a rod lens system or a glass fiber. In addition to this guide bore, the bridge has at least one working channel through which working instruments can be guided. These working instruments can be wires, rods or hoses, for example, that lead to tools arranged in a distal end of the instrument. This at least one working channel can be arranged parallel to or at an angle to the guide bore. Embodiments are conceivable in which the bridge has two or more working channels. At a distal end, the bridge can be coupled to a shaft. This coupling can be carried out, for example, via a bayonet-like rotary clamping ring or an automatic clamping ring. In an instrument of the type in question, the shaft, which is coupled to the bridge, is designed in such a way that it accommodates both the optical unit and the at least one working instrument, which are guided through the bridge. Within the shaft, the instruments and the optical unit are guided as far as the distal end in order to be used at the site of treatment within the body. For example, a telescope for the optical unit can be secured at a proximal end of the bridge.

The present invention provides that the guide bore is segmented from the proximal end to the distal end of the bridge and that the segments have longitudinal axes offset with respect to one another. This segmentation and the offset of the longitudinal axes has the effect that the optical unit is not routed coaxially within the bridge. That is to say, the optical unit can be secured within the bridge in such a way that it is guided into the shaft at an angle with respect to the longitudinal axis of the shaft. This compensates for tilting that occurs during the coupling between the shaft and the bridge, so that in particular the distal end region of the shaft remains free for the further working instruments. The correction taking place in the bridge, and correcting the relative positioning of the optical unit within the shaft, has the result that both the optical unit and the working instruments can be used without restriction at the distal end of the handheld surgical instrument.

The invention preferably provides that at least one segment of the guide bore has a different diameter than at least one other segment. The offset of the longitudinal axes of the various segments causes the relative orientation of the optical unit in the segments to shift. As a result, the optical unit may require more space, at least locally, in order not to abut the wall of the guide bore.

In particular, the invention provides that the guide bore has at least two segments. A particular embodiment may provide that the guide bore has three segments, namely a distal first segment, a middle second segment and a proximal third segment. These segments are all designed in such a way that the optical unit can be guided through the bridge. Likewise, these segments are designed in such a way that they interfere with the coaxial guidance of the optical unit within the guide bore and thereby compensate for the tilting of the optical unit. It is also conceivable that further segments are inserted into the guide bore in order to achieve the aforementioned effect. These segments can be sections within the bore of a few millimeters to centimeters in length, or rings that are arranged inside the bore and act on the rod-like optical unit. For example, it is conceivable that the guide bore has two or three rings, which have a different inner diameter and are offset with respect to a longitudinal axis of the guide bore.

In a preferred embodiment of the invention, provision can be made that the second segment has a larger diameter than the first and the third segment. The offset of the longitudinal axes of the segments causes the rod-like optical unit to tilt or bend locally. As a result of this bending, the optical unit requires an adapted cross section in the guide bore in certain areas so that the optical unit does not abut an inner wall of the hole and change the correction that has been made. Alternatively, it is also conceivable that the second segment has a larger axial offset compared to the first and the third segments.

In a particularly advantageous embodiment of the invention, provision can be made that the diameter of the second segment is 4.0 mm to 5.0 mm, preferably 4.5 mm, the diameter of the first segment is 3.8 mm to 4.4 mm, preferably 4.22 mm, and the diameter of the third segment is 3.8 mm to 4.2 mm, preferably 4.13 mm. These dimensions are such that the third segment encloses the rod-like optical unit with a form fit, preferably over the entire length in the third segment. Thus, the optical unit is fixed in this segment and is given a defined holding point. The diameter of the first segment in turn is dimensioned in such a way that, in the interaction with the offset, a section of the inner wall serves as a support for the optical unit. This means that in this segment the longitudinal axis of the optical unit does not coincide with the longitudinal axis of the segment. Ultimately, the optical unit in this first segment experiences the tilting relative to the longitudinal axis of the shaft. In addition, a second holding point is defined by a support, in particular in order to keep the distal end of the optical unit in a defined position.

Provision can also be made that the longitudinal axis of the first segment is offset, in particular in parallel, with respect to the longitudinal axis of the optical guide of an optical plate. In a particular embodiment of the invention, provision is made that the longitudinal axis of the first segment is offset with respect to the longitudinal axes of the third and/or second segment, in particular in parallel, by 0.1 mm to 0.3 mm, in particular by 0.21 mm. This shift of the longitudinal axis of the first segment with respect to the longitudinal axes of the third and/or second segment can be oriented away from the working channel or toward the working channel. By the interaction of the offset of the longitudinal axes and the different diameters of the segments, a tilting of the optical unit relative to the longitudinal axis of the shaft can be achieved. This tilting ensures that all of the working instruments and the optical unit can be used without their functionalities being restricted.

The invention can also provide that the second segment and the third segment are oriented coaxially. Depending on the application and/or on the design and size and also shape of the instrument, the segments can be designed in different ways. Thus, it is ultimately conceivable that there is an offset between almost every segment and that the diameters of the segments are chosen in such a way that the optical unit is optimally tilted.

A handheld surgical instrument, in particular an endoscope, a cystoscope, a resectoscope or the like, for solving the stated problem has the features of claim 12. Accordingly, provision is made that the handheld surgical instrument has a main body according to at least one of claims 1 to 11.

A preferred embodiment of the invention is explained in detail below with reference to the drawing, In the drawing:

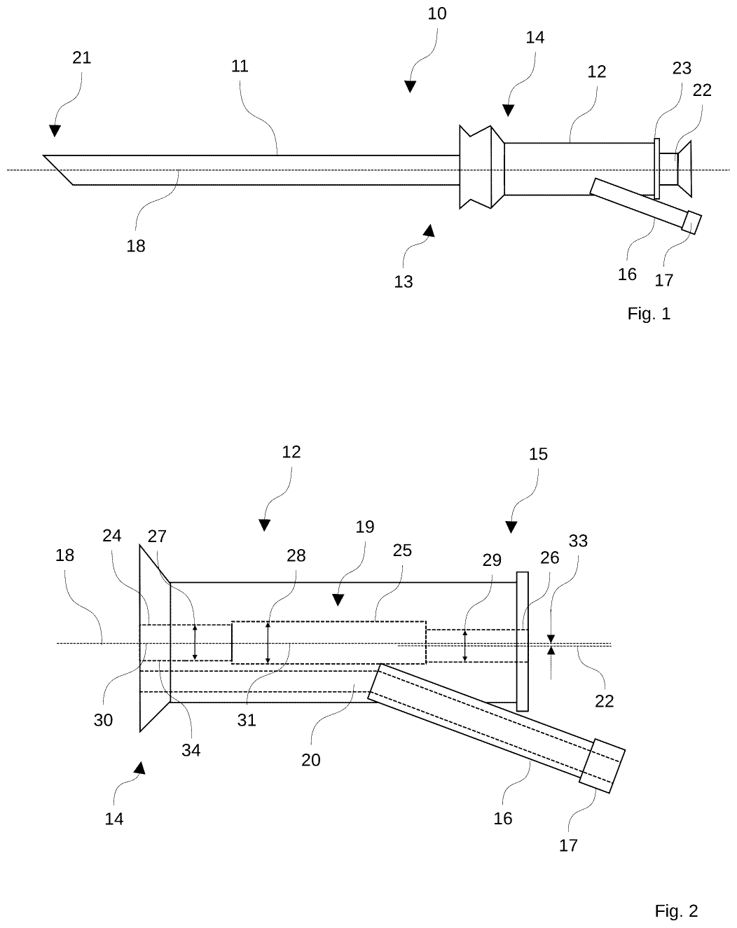

FIG. 1 shows a schematic view of a handheld surgical instrument, and

FIG. 2 shows a cross-sectional view through a bridge.

A handheld surgical instrument 10 is shown in a highly schematic form in FIG. 1. This instrument 10 can be, for example, a cystoscope, a resectoscope or a similar instrument for minimally invasive treatment of a patient. The handheld surgical instrument 10 described here consists essentially of a tubular shaft 11 and a bridge 12. For the treatment of the patient, the instrument 10 is guided with a distal end 21 of the shaft 11 into an opening in the patient's body. The bridge 12 is located outside the body. By way of the bridge 12, various working instruments can be inserted into the body through the shaft 11. In addition, the treatment can be observed or controlled via the bridge 12 and the shaft 11 by means of an appropriate optical unit.

The shaft 11 can be coupled with a proximal end 13 to a distal end 14 of the bridge 12. For example, a screw cap, a click-on cap, a bayonet cap or a clamping ring can be used for this purpose. A telescope 22 with an optical unit can be arranged at a proximal end 15 of the bridge 12. Furthermore, in the embodiment of the instrument 10 shown here, a connection 16 facing downward at an angle is located on the side of the bridge 12. This connection 16 is tubular and can have a valve 17 at a free end.

Inside the bridge 12 there are at least two channel-like passages, which extend at least largely parallel to a longitudinal axis 18 of the handheld surgical instrument 10 and of the shaft 11, namely a guide bore 19 and at least one working channel 20. Also, the guide bore 19 and the working channel 20 extend largely parallel to each other and to the longitudinal axis 18 and run from the proximal end 15 to the distal end 14 of the bridge 12.

The connection 16 represents a continuation of the working channel 20. When the valve 17 is open, various working instruments, such as wires, probes, clamps or the like, can be inserted into the handheld surgical instrument 10 through this tubular channel 20. The working instrument (not shown) is guided through the bridge 12 and through the shaft 11 to a distal end 21 of the shaft 11. The treatment of the patient takes place in front of the distal end 21 of the shaft.

The guide bore 19 serves to accommodate an optical unit (not shown). For example, this optical unit can be a rod lens system (not shown). This rod lens system extends from the proximal end 15 to the distal end 14 of the bridge 12 and is guided further through the entire shaft 11 to the distal end 21 of the shaft 11 and is directed there to the region that is to be treated. At the proximal end 15 of the bridge 12, the optical unit or the rod lens system can be connected to the telescope 22. The telescope 22 and/or the rod lens system can be attached to an optical plate 23 of the bridge 12. An eyepiece or a camera can be attached to the telescope 22, such that the region to be operated on is visible to the surgeon via the rod lens system. As an alternative to the rod lens system, fiber optics can also be used as imaging means.

In the invention described here, the guide bore 19 is segmented. According to the embodiment of the bridge 12 shown in FIG. 2, the guide bore 19 has three segments, namely a first segment 24, a second segment 25 and a third segment 26. While the first segment 24 is aligned with the distal end 14 of the bridge 12, the third segment 26 adjoins the proximal end 15 of the bridge 12. The second segment 25 is located between the first and third segments 24, 26. As an alternative to the embodiment of the bridge 12 shown here, it is also conceivable that the guide bore 19 is divided into two segments or into more than three segments.

The diameters 27, 28, 29 of the three segments 24, 25, 26 are indicated here by double arrows and differ from one another. While the diameter 28 of the second segment 25 is the largest, the diameter 29 of the third segment 26 is dimensioned precisely so that the optical unit can be guided through with a form fit. In the embodiment shown here, the diameter 27 of the first segment 24 is smaller than the diameter 28, but greater than the diameter 29. In a preferred embodiment, the diameter 28 of the second segment is 25 4 mm to 5 mm, preferably 4.5 mm. The diameter 27 of the first segment 24 is 3.8 mm to 4.4 mm, preferably 4.22 mm, and the diameter 29 of the third segment 26 is 3.8 mm to 4.2 mm, preferably 4.13 mm. However, it should be expressly noted that the sizes of the diameters may also assume other values.

The longitudinal axes 30, 31 and 32 of the segments 24, 25, 26 are oriented parallel to one another and parallel to the longitudinal axis 18 of the bridge 12 or of the shaft 11 or of the instrument 10. However, the longitudinal axis 30 of the first segment 24 is offset with respect to the longitudinal axis 32 of the third segment 26 and/or with respect to the longitudinal axis 31 of the second segment 25. The offset 33, indicated by arrows in FIG. 2, between the longitudinal axis 30 and the longitudinal axis 32 can be 0.1 mm to 0.3 mm, in particular 0.21 mm. However, it is also conceivable that the offset 33 between said axes is greater or smaller. The decisive factor for the embodiment shown here is that the longitudinal axis 30 of the first segment 24 is offset upward with respect to the longitudinal axis 32 of the third segment 26, i.e. away from the working channel 20. The offset 33 and the form-fit connection between the third segment 26 and the optical unit have the effect that the optical unit within the first segment 24 rests on a lower portion, a support 34, of the inner wall, and thus a distal end of the optical unit, located at the distal end of the shaft 11, is pushed upward, i.e. away from a working tool. The spacing between the optical unit and the working tool at the distal end means that the optical unit and the working tools do not interfere with each other in terms of their functionality. The widened second segment 25 serves to accommodate a slight bending of the optical unit, specifically without the optical unit abutting an inner wall of the second segment 25.

### LIST OF REFERENCE SIGNS

10 handheld surgical instrument
11 shaft
12 bridge
13 proximal end
14 distal end
15 proximal end
16 connection
17 valve
18 longitudinal axis
19 guide bore
20 working channel
21 distal end
22 telescope
23 optical plate
24 first segment
25 second segment
26 third segment
27 diameter
28 diameter
29 diameter
30 longitudinal axis
31 longitudinal axis
32 longitudinal axis
33 offset
34 support

1. A bridge for a handheld surgical instrument, with a channel-like guide bore for an optical unit and at least one working channel for receiving at least one working instrument, wherein a distal end of the bridge can be coupled to a shaft for the optical unit and the at least one working instrument and a proximal end can be coupled to a telescope, wherein the guide bore is segmented from the proximal end to the distal end, and the segments have longitudinal axes offset with respect to one another.

2. The bridge for a handheld surgical instrument as claimed in claim 1, wherein at least one segment has a different diameter than at least one further segment.

3. The bridge for a handheld surgical instrument as claimed in claim 1, wherein the guide bore has at least two segments.

4. The bridge for a handheld surgical instrument as claimed in claim 1, wherein the guide bore has three segments, namely a distal first segment, a middle second segment and a proximal third segment.

5. The bridge for a handheld surgical instrument as claimed in claim 4, wherein the second segment has a larger diameter than the first and the third segment.

6. The bridge for a handheld surgical instrument as claimed in claim 4, wherein the diameter of the second segment is 4.0 mm to 5.0 mm, the diameter of the first segment is 3.8 mm to 4.4 mm, and the diameter of the third segment is 3.8 mm to 4.2 mm.

7. The bridge for a handheld surgical instrument as claimed in claim 4, wherein the longitudinal axis of the first segment is offset with respect to the longitudinal axis of an optical guide in an optical plate.

8. The bridge for a handheld surgical instrument as claimed in claim 4, wherein the longitudinal axis of the first segment is offset with respect to the longitudinal axes of the third and/or second segment, by 0.1 mm to 0.3 mm.

9. The bridge for a handheld surgical instrument as claimed in claim 4, wherein the second segment and the third segment are oriented coaxially.

10. The bridge for a handheld surgical instrument as claimed in claim 7, wherein the longitudinal axis of the first segment is offset away from the working channel with respect to the longitudinal axes of the third and/or second segment.

11. The bridge for a handheld surgical instrument as claimed in claim 1, wherein a section of an inner wall of a distal section of the guide bore serves as a support for the rod-like optical unit, and an inner wall of a proximal section of the guide bore encloses the rod-like optical unit with a form fit.

12. A handheld surgical instrument with a bridge as claimed in claim 1.
